(19) United States
(12) Patent Application Publication
(10) Pub. No.: US 2005/0014248 A1
(43) Pub. Date: Jan. 20, 2005

(54) Device for analysing a sample in particular by flow cytometry

(76) Inventor: Michel Canton, Levallois Perret (FR)

Correspondence Address: Henderson Farabow;Garrett & Dunner, 1300 I Street N W, Washington, DC, 20005, US

(21) Appl. No.: 10/485,505

(22) PCT Filed: Aug. 2, 2002
(86) PCT No.: PCT/FR02/02791

(30) Foreign Application Priority Data
Aug. 2, 2001 (FR) 0110380

(51) Int. Cl. C12M 1/34; G01J 3/42; G01J 3/427

(52) U.S. Cl. 435/287.2; 356/319

(57) ABSTRACT

The invention concerns a device for analysing a sample comprising a sample receptacle (8) and a mirror (38), the mirror (38) comprising a break (40) such that a light beam (42) can pass through the mirror to reach the receptacle (8). The invention also concerns an assembly for analysing a sample comprising a device (2) and an equipment having a removable housing for receiving the device and a light source, the source being adapted to emit a light beam (42) passing through the mirror (38) to reach the receptacle (8) when the device is in the housing.

The invention relates to devices for analysis of a sample, particularly by flow cytometry.

Flow cytometry is now routinely used in a wide variety of fields (medicine, food processing, biotechnologies, environment), for the analysis of various components such as cells, macromolecules, etc. For example, flow cytometry can be used as follows.

The cells (particulate materials) to be analysed are centred by a liquid stream system along the line of a liquid jet. They pass individually (a few thousands per second) through a laser beam focused on the centre line of the jet and thus induce a number of light signals.

Appropriate optical systems retrieve scattered laser light in a solid angle between 0.5° and 15° (axial scatter) and light emitted perpendicular to the jet and to the laser beam (perpendicular scatter, fluorescence). A set of mirrors and optical filters decomposes collected signals according to their wavelength (for example green and red fluorescence of acridine orange). Optical signals transformed by photodetectors into electrical signals are processed in specialised electronic circuits that assign intensities of emitted signals to each cell. Single or multi-dimensional frequency distributions (histograms) give the population distribution of the analysed cells.

For example, a device of this type is shown in document FR-2-325 038.

In order to make this technique more flexible in use, discardable devices are now available with a shape similar to the shape of a credit card and that can hold a sample to be analysed. This type of device can be fitted removably into the apparatus that includes the light source, optical devices and means of analysing light rays emitted by the sample, and the fluid system capable of entraining the sample. The system is connected to the card to entrain the sample through a card analysis window.

However, one disadvantage with some of these removable cards is that the quantity of light collected for the analysis is very much less than the quantity collected in a conventional device. In a conventional device, optical means such as lenses are usually arranged very close to the analysis stream so as to collect the maximum possible quantity of light, in other words at the largest possible solid angle around the point in the stream through which the incident ray passes. However, in a removable card, the removable nature of the card makes it necessary to provide a minimum space between the stream and the lens, which reduces the solid collection angle such that less light is collected.

To overcome this disadvantage, document EP-1 058 939 proposes that an optical means such as a collection lens should be included in the wall of the card. Since this lens can be placed very close to the stream, the quantity of collected light is increased. This document also proposes to integrate a reflecting element into the card. This type of element can contribute to increasing the collection of light.

One purpose of the invention is to further increase the quantity of light that can be collected within the scope of an analysis device.

Consequently, the invention divulges a device for the analysis of a sample by means of a light ray comprising a sample receptacle and a mirror, the mirror having a discontinuity such that a light ray can pass through the mirror to reach the receptacle.

Thus, by forming a discontinuity in the mirror for passage of the incident ray, the mirror can be extended over a large area particularly on each side of the beam in order to further increase the amount of light collected. This advantage is particularly useful in flow cytometry analysis cards, particularly if the mirror is integrated into the card. It is also useful in cytometry systems without a removable means or even in domains other than cytometry analysis.

The device according to the invention may also have at least one of the following characteristics:
the mirror is fixed to the receptacle,
the device comprises an external wall forming the mirror,
the mirror is fixed on an external wall of the device,
the shape of the device is essentially flat,
the receptacle extends parallel to a main face of the device, the mirror extending adjacent to the receptacle along a thickness e of the device,
the discontinuity is adjacent to the receptacle along a thickness e of the device,
the discontinuity is arranged such that the light ray is inclined with respect to a thickness e of the device,
the device is arranged such that a fluid flows in the receptacle along a predetermined direction, the discontinuity being located in the upstream half of the mirror with reference to the flow direction,
the device comprises a substrate on which one face forms the mirror, this face being in contact with a medium different from the substrate medium and extending between the substrate and the receptacle,
the face forms an internal cavity in the device,
the device comprises a substrate with a face forming the mirror and extending in contact with a medium different from the substrate medium, the substrate extending between the face and the receptacle,
the medium is a gas,
the mirror is in the shape of a portion of a sphere,
the device is arranged such that the ray emerges from the device on one side of the receptacle opposite the mirror,
the device also includes an optical means fixed to the receptacle,
the receptacle is located between the mirror and the optical means,
the device comprises an external wall forming the optical means,
the shape of the receptacle is elongated,
the device comprises a fluid reservoir,
the sample includes a fluid, and
it is an analysis device based on flow cytometry.

The invention also divulges an assembly for the analysis of a sample comprising a device and an apparatus with a reception housing removable from the device and a light source, the source being capable of emitting a light ray through the mirror to reach the receptacle when the device is in the housing.

This arrangement provides a means of checking that the card is correctly positioned in its housing, by measuring the intensity of the incident ray transmitted through the card, which must be maximum, and the intensity of the ray reflected by the periphery of the orifice towards the source, which must be minimum.

Advantageously, the apparatus comprises means of analysing the ray arranged such that the device extends between the source and the analysis means when the device is in the housing.

Advantageously, the assembly comprises means of analysing radiation arriving directly or indirectly from the source so as to determine if the device is fitted at a predetermined position in the housing.

Other characteristics and advantages of the invention will become clearer in the following description of three preferred embodiments given as non-limitative examples.

In the attached drawings:

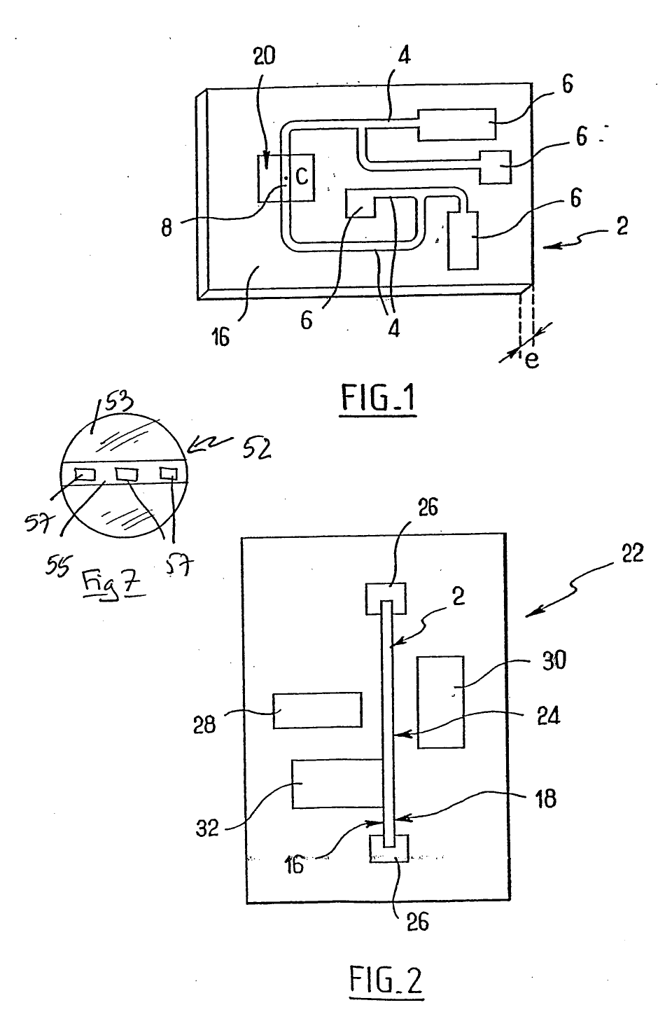

FIG. 1 is a perspective view showing the principle of a device according to the invention,

FIG. 2 is a diagrammatic view of an assembly according to the invention in which the device in FIG. 1 can be fitted,

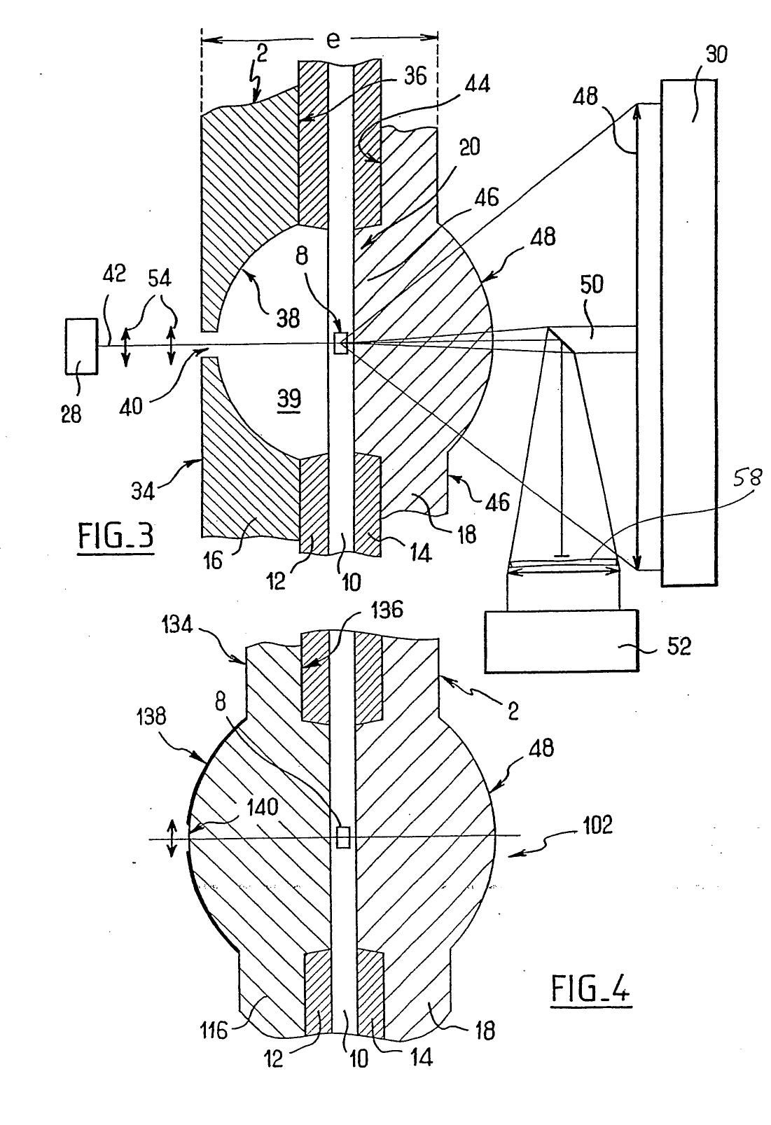

FIG. 3 is a cross-sectional view showing the edge of the device in FIG. 1, and illustrating the first preferred embodiment;

FIG. 4 is a figure similar to FIG. 3 in which identical elements are not shown, and illustrating the second preferred embodiment;

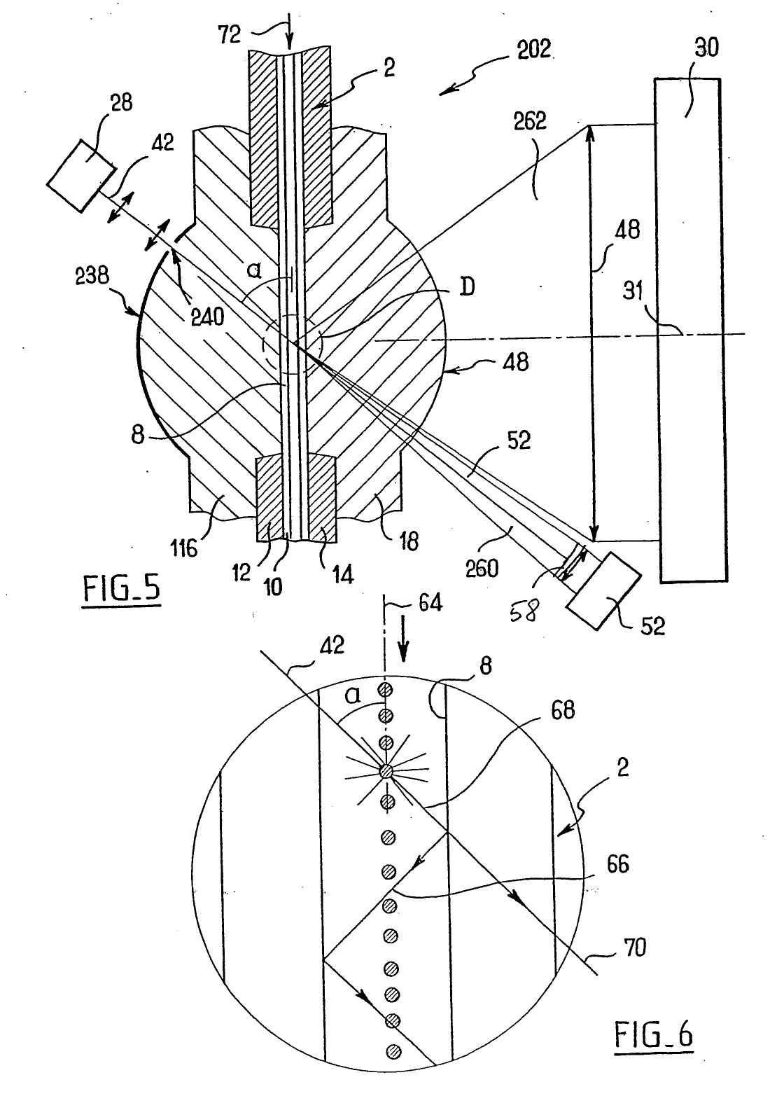

FIG. 5 is a view similar to FIG. 3 illustrating the third preferred embodiment;

FIG. 6 is a larger scale view of detail D in FIG. 5; and

FIG. 7 is a view of the device 52 in FIG. 3.

FIG. 1 shows a device according to the invention in the form of a card 2. This card is generally rectangular and its length and width are similar to the length and width of a credit card, each being of the order of a few centimetres. Its thickness e is of the order of a few millimetres, for example 3 mm. This card is preferably transparent. There are different ducts 4 and one or more compartments 6, one or more reservoirs 6 and/or one or more orifices 6 arranged to be in mutual fluid communication through the ducts 4, all within this thickness. The card may thus contain one or more liquids stored or in circulation inside the card. The card orifice(s) are used to act on the inside of the card to introduce a liquid into the card, to extract a liquid from the card or to circulate a liquid in the card. The card comprises a duct 8 in the form of a capillary in communication with the other ducts 4.

With reference to FIG. 3, the card comprises an internal wall 10, a front intermediate wall 12 and a back intermediate wall 14 extending on each side of the internal wall 10. Therefore, this internal wall is sandwiched between the intermediate walls. The card also comprises a front external wall 16 and a back external wall 18 extending on each side of the group of three walls 10, 12 and 14. The shape of all the five walls 10 to 18 is practically the same, usually the same shape as the card.

With respect to the capillary 8, the intermediate walls 12 and 14 have cutouts facing each other, forming a window 20 through the thickness of the card.

Conventionally and in a manner not described here, the walls 10, 12 and 14 are formed by stacking several sheets each with internal cutouts adapted such that the stack of the different sheets defines the above-mentioned elements 4, 6, 8 and 20 inside the card. The principle of this type of card is known.

The card 2 will fit removably into an apparatus 22 like that shown diagrammatically in FIG. 2. Consequently, the apparatus is provided with a housing 24 formed by adapted means 26. Conventionally, in a manner known in itself in principle, this apparatus comprises at least one light source 28 such as a laser radiation source, and means 30, 52 for analysing and processing radiation output from the card. Conventionally, these means include different optical devices such as lenses, filters, mirrors, etc., and devices capable of converting this radiation into electronic form for processing purposes. The apparatus also comprises means 32 necessary to circulate one or more fluids inside the card. In particular, these means may comprise one or more pumps. Once the card is placed in its housing, these means are capable of coming into fluid communication with the inside of the card through the orifices in it.

With reference to FIG. 3 that shows a section perpendicular to the general plane of the card, the front wall 16 of the card 2 comprises a plane external face 34 designed to extend facing the source 28 when the card fits into the housing 24. The internal face 36 forming the second main face of this wall is also plane over most of its surface, but there is a hollow spherical portion 38 forming a cavity 39.

This spherical portion 38 extends facing the capillary 8 and the window 20. This portion is coated with an adapted coating such as a metal coating in order to form a mirror. Therefore, this mirror forms the dioptre forming the junction between the material of the front wall 16 and the air filling in the cavity 39. The mirror is formed such that the point c of the capillary extending to the centre of the window 20 occupies the centre of the sphere.

In this example, an orifice 40 is formed in the front wall 16, in the area of the mirror 38 extending adjacent to the capillary 8 along the thickness e. Considering the shape of the mirror, this area is the part of the wall 16 in which it is thinnest. In the apparatus 22, the source 28 is positioned such that when the card 2 is located in the housing 24, a ray 42 emitted by the source passes through the wall 16 at the orifice 40 to intercept the capillary 8 at the centre c of the window 20, in other words at the focus of the mirror 38.

The back wall 18 has an internal face 44 with a protuberance 46 capable of filling in the cutout in the back intermediate wall 14 corresponding to the window 20. The back wall 18 also has a back external face 46. The largest part of this face is formed by a plane area. It also comprises a convex spherical area 48 projecting from the plane area. This area 48 forms a lens extending facing the window and the focus of which is located at the point of the capillary 8 at which the incident ray 42 will arrive. Therefore, the capillary 8 is inserted between the mirror 38 and the lens 48. The lens 48 is a convergent lens. In the embodiment shown in FIG. 3, the incident ray 42 is essentially perpendicular to the plane of the card.

The apparatus also comprises means such as a mirror 50 arranged close to the processing means 30, along the path of the incident ray 42 emitted by the source 28 and capable of deviating the fraction of the incident ray that passed through the capillary towards an appropriate device not shown, without having been significantly deviated by the capillary. Consequently this fraction, that we will call the transmitted fraction, includes practically no useable information and therefore has to be eliminated without hindering the analysis of other fractions.

During operation, when the card 2 fits into the housing, the incident ray 42 of the source 28 passes through the orifice 40 and strikes the capillary 8 containing the sample to be analysed at a point that corresponds to the centre of the mirror 38 and the focal point of the lens 48.

The transmitted fraction of this ray passes through the card and is eliminated by the mirror 50.

Another part of this ray is diffracted during interactions with particulate materials of the sample as it passes through the capillary. This diffraction takes place at a solid angle of a few steradians on each side of the transmitted beam. This part contains light information about the size of particulate materials in the sample and can therefore be usefully analysed. This fraction passes through the rear wall 18. The rays that compose it are initially divergent. Their orientation is modified at the passage of the lens 48 that for example transforms this fraction into parallel radiation or slightly divergent radiation. In FIG. 3, the function of the lens 48 has also been shown by the arrow 48 according to the conventional symbol. Therefore the entire beam, initially diffracted at a small solid angle (typically between 0.5 and 15 steradians), is collected to be directed to processing means 52 through the mirror 50.

FIG. 7 shows a view of the front face 53 of the means 52 that collects the radiation. This face is generally circular in shape. It is provided with a laser occulting bar 55 at mid-height extending over the entire width of the face. This bar comprises direct laser light intensity detectors 57 (three in this case) at intervals from each other.

A fraction of incident ray 42 is also scattered at a large solid angle about the incidence point of the ray on the capillary, and even more frequently in the entire space, in other words on a solid angle of 4 steradians. Part of this fraction scattered towards the lens emerges from the card through the lens 48 such that it is also collected and sent to the processing means 30. Another part of this scattered fraction is oriented towards the mirror 38 that reflects it to the centre of the capillary, which means that it will be collected by the lens 48 and then by the processing means 30. Therefore a very large part of the scattered beam is therefore sent directly or indirectly to the processing means 30.

The components forming the scattered beam may be separated from each other and measured, for example, at the wavelength of the laser and at the fluorescence length(s) characteristic of fluorescent markers used for the purpose of the analysis and resulting from the passage of the ray in the capillary.

The assembly consisting of the apparatus 22 and the card 2 may comprise optical devices 54 arranged between the source 28 and the orifice 40, for example convergent lenses capable of focusing the incident ray on the capillary. These devices may either be fixed permanently on the apparatus or fixed permanently onto the card 2, being integrated into the card.

The different components of the diffracted and scattered beams can be separated by means of conventional devices integrated into the apparatus such as separating mirrors, filters, networks or prisms. Each component can be quantified by means of photodetectors with an appropriate sensitivity.

The mirror 38 provides a means of collecting back scattered light and reflecting it superposed with scattered light directly towards the lens, forwards so that the flux intensity can be doubled.

The orifice 40 makes the area of the mirror large without the incident ray forming an obstacle. The presence of the orifice 40 in the mirror 38 also provides a means of verifying that the position of the card 2 in its housing 24 is correct. For example, it will be possible to provide means 58 between the mirror 50 and the analysis means 52 as shown (or even within the means 52 themselves), to measure the intensity of the light flux reflected by the mirror 50 and received by the device 58. Means can also be provided at the source 28 to measure the intensity of a light flux formed by the fraction of the incident ray 42 reflected by the periphery of the orifice 40. When the position of the card in its housing is correct, the light flux received by the device 58 should be maximum while the light flux reflected by the periphery of the orifice to the source 28 will be minimum. On the other hand, if the card is not in the right position, the orifice 40 is no longer precisely facing the source 28. Under these conditions, the light intensity received by the device 58 is not maximum, while the intensity reflected by the periphery of the orifice towards the source is not minimum. Naturally, control over the position of the card in the apparatus may be automated for use in an auto-optimisation system.

In fact, the shape of the section through the laser beam at the illumination point c is a flattened ellipse, with a major axis (contained in the plane in FIG. 3) with a length of between 60 and 100 micrometers, or possibly between 20 and 100 micrometers, and a minor axis (perpendicular to the plane in the figure) with a length between 10 and 20 micrometers, or possibly between 3 and 20 micrometers. The dimensions of cells or particulate materials being analysed by cytometry can be between 0.1 and 20 micrometers.

The largest transverse dimension of the capillary 8 through which the ray passes, and possibly its diameter if its cross-section is circular, may be of the order of 100 micrometers. It may also be between 50 micrometers and 1 millimetre. As shown in FIG. 3, in this case the cross section of this duct 8 is rectangular in a plane perpendicular to its longitudinal axis. In this case the width of this section is between 20 and 200 μm and its length is between 50 μm and 1 mm.

The sheets forming the internal part of the card, particularly forming the wall 10, could be composed of a material known under the name mylar. The intermediate walls and the external walls may be made of glass or a plastic material such as PMMA or polycarbonate.

The source 28 may be low power (for example between 10 milliwatts and 100 milliwatts, or between 1 mW and 25 mW), which can reduce the cost. Another advantage of the low power is that it simplifies means to be implemented to deviate and absorb the transmitted ray (devices 50 and 58) and to eliminate any parasite rays.

In one variant embodiment, the cavity 39 can be filled with a material with a refraction index different from the index of air. For example, this material may be a gel known in itself for its advantageous index. This type of material can improve the orientation of rays as they are reflected in the cavity.

FIG. 4 shows a second preferred embodiment of the invention. The apparatus is identical to the apparatus in the first mode. All that changes is the card 102. The shape of the back wall 18 is the same as in the mode in FIG. 3. The front wall 116 is generally symmetric with the back wall 18 about the median plane extending through the thickness of the card. In other words, in this case the internal face 136 has a protuberance that fills the window 20. Furthermore, the external face 134 comprises a spherical portion 138 projecting from the plane area of this face. The portion 138 is coated with a coating such as a metallisation to confer reflecting properties to it so as to make it a mirror. Therefore, the mirror 138 is formed by the dioptre forming the junction between the wall 116 and ambient air. However, unlike the arrangement of the embodiment in FIG. 3, the wall 116 is inserted between ambient air and the capillary 8, while in the previous embodiment, the cavity full of air was inserted between the wall and the capillary. Therefore, this embodiment is different from the previous embodiment particularly in that in this case the coating 138 extends outside the card, and no longer inside it.

There is a discontinuity 140 in the coating 138 adjacent to the point c of the capillary 8 occupying the centre of the window 20 along the thickness of the card. This discontinuity extends to the same location as the orifice 40 in the embodiment in FIG. 3.

The operation is approximately the same as in the previous mode. Thus, the beam output from the source 28 penetrates into the card through the area 140 of the uncoated wall 116 to strike the capillary 8. The axial fraction is transmitted as before to devices 52 and 58. The fraction diffracted at small angles passes through the lens and is then processed by the means 52. Finally, the fraction scattered at large angles passes through the lens and is processed by means 30 while a back scattered fraction passes through the wall 116 and is reflected by the mirror 138 that, like the fraction in the first mode, is centred on the capillary. Therefore, the rays are reflected towards the focal point of the lens, collected by the lens and then processed by the means 30.

However, these two embodiments have the disadvantage that the transmitted fraction follows the same path as a significant part of the fraction scattered at large angles. Consequently, eliminating the transmitted fraction to avoid disturbing the analysis concomitantly eliminates this part of the scattered fraction. Furthermore, parasite reflections of the incident ray on the walls of the capillary and the walls of the card remain along the path of the measured rays and can disturb the analysis.

The embodiment that will now be described with reference to FIGS. 5 and 6 is better in this respect.

This embodiment is very similar to the embodiment in FIG. 4 in which the discontinuity on the mirror is formed by an interruption to the coating forming the external mirror. In this third embodiment, the discontinuity 240 of the card 202 does not extend as far as the centre c of the window along the direction of the thickness e, but is offset towards an edge of the mirror as shown in FIG. 5. Thus, the straight line extending from this discontinuity 240 towards the centre c of the window occupied by the capillary is inclined at an acute angle with respect to the longitudinal direction of the capillary.

In the apparatus, the position of the source 28 is modified accordingly. In this case, the axis of the source intersects the axis 31 of the processing means 30 at the centre c of the window. The source no longer extends directly towards the means 30, nor coaxially with them. The source is arranged such that when the card is correctly positioned in its housing, the ray 42 passes through the discontinuity 240 to reach the centre c of the window in the capillary. Similarly, the device 52 that will receive a fraction of the diffracted ray no longer extends towards the means 30 coaxially with them, but is aligned with the source, the discontinuity 240 and the centre c of the window.

Therefore, a central area of the very large area lens is located facing the means 30 without any obstacles between them such that all rays passing through this area can be collected for processing. This arrangement is such that the solid angle 260 corresponding to the fraction of the ray collected by the devices 52 and 58 extends entirely outside the solid angle 262 corresponding to the fraction of radiation (large angles) collected by the means 30.

FIG. 5 shows a section of the card in a plane perpendicular to the general plane of the card. The capillary extends in the plane of the section, unlike the cases in FIGS. 3 and 4. In this figure, it can be seen that the source 28 is arranged such that the ray 42 extends in this plane. It is not strictly necessary that the ray extends in this plane. Thus, the solid angles 260 and 262 can be separated by placing the source 28 outside the plane in FIG. 5, while providing the discontinuity 240 close to an edge of the mirror 238. However, the arrangement of the source 28 in this plane means that a large and very significant fraction of the scattered and diffracted beam can be collected.

Moreover, it is advantageous if the angle a shown in FIG. 6 formed between the incident ray 42 and the axis 64 of the capillary, is significantly less than 90° such that at least a large part 66 of the transmitted beam 68 remains trapped in the capillary 8 by reflection on the internal face of the capillary, the capillary thus forming a wave guide. It follows that the intensity of the fraction 70 of the transmitted beam effectively emerging from the card is smaller and is therefore easier to eliminate. This arrangement also makes it possible to significantly reduce parasite radiation generated during the analysis. In the embodiment in FIG. 5, the angle a is equal to 45°. It will advantageously be between 30 and 50°.

Moreover, it is preferable if the source 28 is brought towards an upstream part of the capillary 8 as shown in FIG. 5, rather than a downstream part of the capillary with reference to the direction of the fluid flow in the capillary shown by the arrow 72. The radiation trapped in the capillary is sent in this way towards a downstream fluid fraction that has already been analysed.

In the opposite arrangement, part of the radiation would be sent to the upstream fractions and could cause an early reaction with fluorochromes present in the liquid or other substances that would interact with incident radiation and consequently could distort the analysis results.

Particulate materials to be analysed such as cells or macromolecules have been shown in FIG. 6 in the form of balls passing one after the other in the capillary 8 in accordance with the principle of flow cytometry.

This embodiment operates very much like the previous embodiments. The incident radiation 42 penetrates into the card through a discontinuity 240 and reaches the capillary at the centre of the window. A fraction 66 of the transmitted radiation 68 remains trapped in the capillary, while a fraction 70 is processed and eliminated by the means 52 and 58. A diffracted fraction 260 of the radiation is collected by the lens 48 and is received by the processing means 52. A fraction of the radiation scattered towards the lens is received by the means 30. The fraction scattered towards the mirror is reflected by the mirror 238 at the centre of the window and is collected by the lens 48 in the same way.

The device 2 could be discardable after a single use. It may be made in large production series. The capillary could be formed by superposition of different sheets located at the centre of the card. Alternately, it could be an individual capillary, for example made of glass or quartz.

The card could comprise one or more optical devices such as mirrors, lenses, prisms, networks, etc. One or more of these means could be made as a single part with one of the external or internal walls of the card. One or more of these means could be added permanently onto the card. In one variant embodiment in FIG. 5, a mirror adjacent to a cavity could be provided as shown in mode in FIG. 3.

The device according to the invention provides a means of collecting a large quantity of light emerging from the capillary. The mirror and the lens are fixed permanently onto the card in the embodiments described above, therefore they are centred once and for all with respect to the capillary during assembly.

In the embodiments in FIGS. 4 and 5, and partly in the embodiment in FIG. 3, the confinement of optical devices around the capillary without the presence of air solves problems related to the presence of parasite dust.

Obviously, various modifications could be made to the invention without going outside the scope of the invention.

The invention may be used during analyses carried out using techniques other than flow cytometry. For example, it could be used for an electrophoresis technique.

Although the embodiments described above have been described with reference to a laser source, the incident radiation could be of various natures. It could be ordinary light, non-laser light, non-coherent light, non-monochromatic light, fluorescent light, ultra-violet light, or a non-luminous or even non-electromagnetic type of wave (sound wave, gamma radiation, X-ray, etc.).

The invention could be used in a device in which samples are fixed and are not removable. The fluid could be static rather than mobile. The fluid could be a non-aqueous fluid such as a solvent or an oil. In some circumstances, the invention can be used without the presence of a fluid. Thus, the invention could be used with a capillary comprising a solid active constituent.

In one variant embodiment shown in FIG. 3, the orifice 40 could be placed near an upstream area of the capillary according to an arrangement of the source 28 similar to that shown in FIG. 5.

Independently of the presence of the discontinuity, a device such as a card could be provided comprising a receptacle and a mirror (for example a spherical mirror) fixed to the device. This device could also comprise an optical device such as a lens arranged such that the receptacle extends between the mirror and the lens in accordance with the illustrated embodiments.

1.-26. (Cancel).

27. Assembly for the analysis of a sample by means of a light ray comprising:
a device comprising a sample receptacle and a mirror; and
an apparatus with a housing for removably receiving the device and a light source,
wherein the mirror has a discontinuity such that the light source can emit a light ray that passes through the mirror to reach the receptacle when the device is in the housing.

28. Assembly according to claim 1, wherein the mirror (38, 138, 238) is fixed to the receptacle.

29. Assembly according to either of claims 1 or 2, wherein the device comprises an external wall (16, 116) forming the mirror.

30. Assembly according to either of claims 1 or 2, wherein the mirror is fixed on an external wall of the device.

31. Assembly according to either of claims 1 or 2, wherein the shape of the device is essentially flat.

32. Assembly according to either of claims 1 or 2, wherein the receptacle (8) extends parallel to a main face (34, 134) of the device, the mirror (38, 138, 238) extending adjacent to the receptacle along a thickness (e) of the device.

33. Assembly according to either of claims 1 or 2, wherein a discontinuity (40, 140) is adjacent to the receptacle along a thickness (e) of the device.

34. Assembly according to either of claims 1 or 2, wherein a discontinuity (240) is arranged such that the light ray (42) is inclined with respect to a thickness (e) of the device.

35. Assembly according to claim 34, wherein a fluid is capable of flowing in the receptacle along a predetermined direction (72), the discontinuity (240) being located in the half of the mirror (238) upstream with reference to the flow direction.

36. Assembly according to either of claims 1 or 2, wherein the device comprises a substrate (16) on which one face (38) forms the mirror, this face being in contact with a medium different from the substrate medium and extending between the substrate (16) and the receptacle (8).

37. Assembly according to claim 36, wherein the face (38) forms an internal cavity (39) in the device.

38. Assembly according to either of claims 1 or 2, wherein the device comprises a substrate with a face (138, 238) forming the mirror and extending in contact with a medium different from the substrate medium, the substrate (116) extending between the face (138, 238) and the receptacle (8).

39. Assembly according to claim 36, wherein the medium is a gas.

40. Assembly according to either of claims 1 or 2, wherein the mirror (38, 138, 238) is in the shape of a portion of a sphere.

41. Assembly according to either of claims 1 or 2, wherein a ray (42) can emerge from the device on the side of the receptacle (8) opposite the mirror (38, 138, 238).

42. Assembly according to either of claims 1 or 2, wherein the device also includes an optical means (48) fixed to the receptacle.

43. Assembly according to claim 42, wherein the receptacle (8) extends between the mirror (38, 138, 238) and the optical means.

44. Assembly according to claim 43, wherein the device comprises an external wall (18) forming the optical means (48).

45. Assembly according to either of claims 1 or 2, wherein the shape of the receptacle (8) is elongated.

46. Assembly according to either of claims 1 or 2, wherein the device comprises a fluid reservoir (6).

47. Assembly according to either of claims 1 or 2, wherein the sample comprises a fluid.

48. Assembly according to either of claims 1 or 2, wherein the device is an analysis device based on flow cytometry.

49. Assembly according to either of claims 1 or 2, wherein the device is a microfluidic device.

50. Assembly according to claim 49, wherein the apparatus comprises analysis means (30, 52) of the ray (42) arranged such that the device (2) extends between the source (28) and the analysis means when the device is in the housing (24).

51. Assembly according to claim 50, which includes means for analyzing radiation arriving directly or indirectly from the source so as to determine if the device is received at a predetermined position in the housing.
